(19) United States
(12) Patent Application Publication
(10) Pub. No.: US 2019/0167242 A1
(43) Pub. Date: Jun. 6, 2019

(54) DEVICES AND METHODS FOR CLOSING A LEFT ATRIAL APPENDAGE

(71) Applicant: Edwards Lifesciences Corporation, Irvine, CA (US)

(72) Inventors: Stanton J. Rowe, Newport Coast, CA (US); Robert S. Schwartz, Inver Grove Heights, MN (US)

(21) Appl. No.: 16/200,455

(22) Filed: Nov. 26, 2018

Related U.S. Application Data
(60) Provisional application No. 62/594,182, filed on Dec. 4, 2017.

(51) Int. Cl. A61B 17/00; A61B 17/12

(52) U.S. Cl. CPC A61B 17/0057; A61B 17/12122; A61B 2017/00632; A61B 2017/00592; A61B 2017/00623; A61B 17/12172

(57) ABSTRACT

Disclosed herein are devices and methods for closing a left atrial appendage (LAA) that use an expandable disk having a deployment anchor and a plurality of peripheral hooks. The LAA closure devices are configured to expand to a diameter that is larger than a diameter of the LAA ostium. The deployment anchor attaches the disk to the tissue of the LAA that is everted or invaginated. The expandable disk causes the everted or invaginated LAA to expand and flatten within the left atrium of the heart. The peripheral hooks pierce the tissue of the LAA and engage the left atrial wall to close the LAA predominantly with its own tissue.

### CROSS-REFERENCE TO RELATED APPLICATIONS

This application claims the benefit of U.S. Provisional Application No. 62/594,182, filed Dec. 4, 2017, the contents of which are incorporated herein in their entirety.

### BACKGROUND

### Field

The present disclosure generally relates to devices and methods for closing the left atrial appendage.

### Description of Related Art

Open heart surgery is associated with a very high incidence of perioperative atrial fibrillation. In valve repair or replacement, the rate of perioperative atrial fibrillation is approximately 45%. In patients with non-valvular atrial fibrillation, embolic stroke is thought to occur from thrombi forming in the left atrium, with the left atrial appendage (LAA) being the principal site of thrombus formation. In atrial fibrillation, the heart's upper chambers, or atria, beat irregularly. Pooling of blood flow in the LAA during atrial fibrillation can increase the risk of blood clot formations that could travel to the brain and cause a stroke. Antiarrhythmic drugs and catheter ablation may be effective in symptomatic relief for patients with atrial fibrillation and the prevention of thromboembolic events may be treated using oral anticoagulation (e.g., vitamin K antagonists, VKA).

The left atrial appendage (LAA) is a small, ear-shaped sac in the muscle wall of the left atrium. Among patients that do not have valve disease, the majority of blood clots that occur in the left atrium start in the LAA. In some circumstances, it may be advantageous to seal off the LAA to reduce a risk of stroke and to reduce or eliminate the need to take blood-thinning medication.

### SUMMARY

In a first aspect, the present disclosure relates to a device for closing a left atrial appendage. The device includes an expandable disk having an expanded diameter that is larger than 10 mm. The device also includes a deployment anchor attached to the expandable disk near a center of a first side of the expandable disk, the deployment anchor configured to puncture a tissue of a left atrial appendage and to anchor the expandable disk to the tissue of the left atrial appendage. The device also includes a plurality of closure anchors attached to the expandable disk near a periphery of the expandable disk, the plurality of closure anchors configured to secure the expandable disk to tissue of a left atrium. The device is configured for delivery in a compact state and expands to an expanded state to cause an everted left atrial appendage to assume a size that is larger than an ostium of the left atrial appendage.

In some embodiments of the first aspect, the closure anchors are attached to the expandable disk on a second side of the expandable disk, the second side opposite the first side. In some embodiments of the first aspect, the closure anchors are attached to the expandable disk on the first side of the expandable disk. In some embodiments of the first aspect, the expandable disk comprises a disk of a nickel titanium braid.

In some embodiments of the first aspect, the device is configured to assume a compact state for delivery and a deployed state for closing a left atrial appendage. In further embodiments, the compact state comprises reducing a diameter of the expandable disk to fit within a sheath of a delivery system. In further embodiments, the deployed state comprises the expandable disk assuming a size and shape having the expanded diameter that is larger than a typical ostium of a left atrial appendage.

In some embodiments of the first aspect, the deployment anchor extends at least 5 mm from the expandable disk. In further embodiments, the deployment anchor extends less than or equal to 15 mm from the expandable disk.

In some embodiments of the first aspect, the deployment anchor comprises at least 3 arms of a self-expanding material. In further embodiments, the deployment anchor comprises less than or equal to 6 arms of the self-expanding material. In further embodiments, the self-expanding material of the deployment anchor comprises nickel titanium.

In some embodiments of the first aspect, the expandable disk includes radial supports. In further embodiments, individual closure anchors are coupled to ends of corresponding radial supports.

In some embodiments of the first aspect, the plurality of closure anchors comprises less than or equal to 6 anchors. In some embodiments of the first aspect, the plurality of closure anchors comprises at least 2 anchors.

In a second aspect, the disclosure relates to a left atrial appendage closure kit including the device of the first aspect and a delivery system having a retractable sheath configured to house the expandable disk in a compact state.

In some embodiments of the second aspect, the delivery system includes a rounded catheter tip. In some embodiments of the second aspect, the delivery system the sheath is configured to be pulled back during operation to release the expandable disk in the compact state such that the expandable disk expands to assume a deployed state. In some embodiments of the second aspect, the delivery system is configured to disengage from the device after the device secures an everted left atrial appendage to a left atrial wall.

In a third aspect, a LAA closure device is provided that includes an expandable disk having an expanded diameter that is larger than 10 mm. The device also includes a deployment anchor attached to the expandable disk near a center of a first side of the expandable disk, the deployment anchor configured to puncture a tissue of a left atrial appendage and to anchor the expandable disk to the tissue of the left atrial appendage. The device also includes a securing ring forming an annulus. The device also includes a plurality of closure anchors attached to the securing ring, the plurality of closure anchors configured to secure the expandable disk to tissue of a left atrium. The expandable disk is configured for delivery in a compact state and expands to an expanded state to cause an everted left atrial appendage to assume a size that is larger than an ostium of the left atrial appendage.

In a fourth aspect, a method for closing a left atrial appendage is provided. The method includes anchoring an expandable disk to an everted tissue wall of the left atrial appendage with a deployment anchor attached to the expandable disk. The method also includes expanding the expandable disk to expand the everted tissue wall to cover an ostium of the left atrial appendage. The method also includes securing the everted tissue wall of the left atrial appendage to a wall of the left atrium with a plurality of closure anchors.

In some embodiments of the fourth aspect, the method also includes everting the tissue wall of the left atrial appendage so that the tissue wall of the left atrial appendage is within the left atrium. In some embodiments of the fourth aspect, everting the tissue wall comprises using a rounded catheter tip from a location external to a heart to evert the left atrial appendage. In some embodiments of the fourth aspect, everting the tissue wall comprises using a rounded catheter tip from a location within the left atrium to evert the left atrial appendage.

In some embodiments of the fourth aspect, the deployment anchor is coupled to a first side of the expandable disk. In further embodiments, the plurality of closure anchors is attached to the first side of the expandable disk. In further embodiments, the plurality of closure anchors is attached to a second side of the expandable disk, the second side opposite the first side. In further embodiments, the plurality of closure anchors is attached to a securing ring. In further embodiments, everting the tissue wall comprises using a rounded catheter tip from a location external to a heart to evert the left atrial appendage and securing the everted tissue wall to the wall of the left atrium comprises applying a force on the securing ring from within the left atrium so that the closure anchors penetrate the everted tissue wall and secure to the wall of the left atrium.

In some embodiments of the fourth aspect, the method is performed during a minimally invasive procedure. In some embodiments of the fourth aspect, the method is performed during open heart surgery. In some embodiments of the fourth aspect, the expandable disk is in a compact state to anchor the expandable disk to the everted tissue wall of the left atrial appendage. In some embodiments of the fourth aspect, the method also includes pulling back a sheath of a delivery system to deploy the expandable disk.

### BRIEF DESCRIPTON OF THE DRAWINGS

Various embodiments are depicted in the accompanying drawings for illustrative purposes, and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements.

FIGS. 1A, 1B, and 1C illustrate various views of an example LAA closure device.

FIGS. 2A, 2B, and 2C illustrate various views of another example LAA closure device.

FIGS. 3A and 3B illustrate various views of another example LAA closure device having a securing ring.

FIGS. 4A, 4B, and 4C illustrate example embodiments of LAA closure devices in a compact state.

FIGS. 5A, 5B, 5C, 5D, 5E, and 5F illustrates steps in a process of installing an example LAA closure device using internal approach.

FIGS. 6A, 6B, 6C, 6D, 6E, and 6F illustrates steps in a process of installing another example LAA closure device using an external approach.

FIGS. 7A, 7B, 7C, 7D, 7E, and 7F illustrates steps in a process of installing an example LAA closure device having a securing ring using an internal and external approach.

FIG. 8 illustrates an example method of installing a LAA closure device.

### DETAILED DESCRIPTION

The headings provided herein are for convenience only and do not necessarily affect the scope or meaning of any of the claimed embodiments.

### Overview

There are many designs for LAA closure devices. They typically fall into two basic categories: plugs and clamps. The plugs use a variety of differently shaped bodies to fill the LAA cavity to close the LAA to thrombus formation. These take many forms from nitinol covered half stents to braided disks. The second form of closure is the clamp, which is applied externally to the appendage during surgery. These approaches frequently leave a “neck” portion of the LAA which remains susceptible to thrombus formation.

LAA closure procedures typically include LAA exclusion with sutures on the epicardial or endocardial surface and LAA excision through staples or removal and oversew. Percutaneous approaches for LAA occlusion include obstruction of the LAA orifice with an occlusion device or percutaneous suture ligation using an endocardial or epicardial approach.

A primary difficulty in closing the LAA is the variations in shape and size of LAAs between subjects. Anatomical studies have described numerous shapes of the LAA, for example, as a long, narrow, tubular, and hooked structure. Four typical LAA morphologies may be described as (1) “chicken wing” where the LAA morphology presents an obvious bend in the proximal or middle part of the dominant lobe, or folding back of the LAA anatomy on itself at some distance from the perceived LAA ostium; (2) “cactus” where the LAA morphology is composed of a dominant central lobe with secondary lobes extending from the central lobe in both superior and inferior directions; (3) “windsock” where the LAA morphology has one dominant lobe as the primary structure, which is larger than the second or distal portions of the LAA; and (4) “cauliflower” where the LAA morphology presents with a main lobe that is not longer than the distal part of the appendage, with more-complex internal characteristics than the chicken wing or windsock morphologies. The shape of the LAA ostium is typically elliptical, with a long diameter ranging from about 10 mm to about 40 mm.

Another difficulty in closing the LAA using obstructions is that the body treats the object as a foreign body, increasing the probability of clotting on the foreign body. This may be particularly disadvantageous for patients in need of LAA closure because these patients are not typically allowed to take anti-coagulation medications.

Accordingly, to address these and other issues, disclosed herein are LAA closure devices and methods that use the tissue of the patient as a primary means of closure, reducing the probability or likelihood of clotting. Furthermore, the disclosed devices and methods flatten and secure an everted LAA against the left atrial wall thereby reducing or eliminating foreign bodies in the flow field of the left atrium. Thus, the closure devices are without significant tissue protrusion and tissue overgrows devices readily. Moreover, by everting and securing the everted LAA to the left atrial wall, the disclosed devices and methods are substantially independent of LAA shape. In addition, the disclosed devices and methods are applicable in minimally invasive surgery or open surgery and can be used in internal, external, or a combination of internal and external approaches.

In particular, disclosed herein are devices and methods that relate to a left atrial appendage closure device that is used to evert the LAA, close it, and secure it to the left atrial wall. Advantageously, the disclosed LAA closure devices can be used during open heart surgery or using a trans-catheter approach. Another advantage is that because the disclosed LAA closure devices predominantly use the everted tissue of the LAA as a closure mechanism, there is little or no need for additional anticoagulation. In addition, because the disclosed LAA closure devices evert the LAA and secure it to the left atrial wall, the disclosed devices work for all or nearly any shape, size, or configuration of LAA.

Embodiments of the LAA closure devices include at least three components: an expandable disk, a deployment anchor attached to the disk, and a plurality of proximal anchors attached to the expandable disk or to a separate securing ring. The expandable disk can be made of a self-expanding material, such as nickel titanium (e.g., Nitinol wire). The self-expanding material can be formed in a braid, in some instances. The deployment anchor is configured to puncture everted LAA tissue and to secure the disk in place. When the disk expands to a size larger than the LAA ostium, the everted LAA tissue flattens and presses against the left atrial wall. When this happens, the plurality of closure anchors can secure the expandable disk to the left atrial wall to close the LAA predominantly with its own tissue.

Advantageously, LAA closure devices are disclosed herein that are easy to use and are effective for use during open heart surgery or using a transcatheter approach. The LAA closure devices advantageously do not require additional anticoagulation because everted LAA tissue is predominantly used as a closure mechanism. The closure devices can be used with a deployment system that utilizes a rounded catheter tip to evert the LAA into the left atrial cavity. Once positioned in the left atrium, a deployment anchor is used to puncture the tip of the everted LAA. Once the deployment anchor is deployed, retraction of a sheath deploys an expandable disk into or onto the everted LAA, creating an expanded body larger in diameter than the LAA ostium, which is typically about 15 mm to about 30 mm in diameter. Attached to the expanded disk are small, closure anchors. The expandable disk can be pushed or pulled toward the left atrial wall so that the closure anchors engage the left atrial wall to secure the everted LAA tissue to the left atrial wall, thereby closing the LAA with its own tissue. The delivery system can then be disengaged and withdrawn from the LAA.

The disclosed LAA closure devices include an expandable disk that has a diameter that is larger than an opening of a typical LAA ostium. Such LAA closure devices can also include a deployment anchor attached to the expandable disk near a center of the first side of the expandable disk, the deployment anchor configured to puncture a tissue of the LAA and to anchor the expandable disk to the tissue of the LAA. Such LAA closure devices can also include a plurality of closure anchors attached to the expandable disk near a periphery of the expandable disk, the plurality of closure anchors configured to secure the expandable disk to tissue of the left atrium. Such LAA closure devices can be configured for delivery in a compact state and can expand to an expanded state for deployment to cause an everted LAA to assume a size and configuration wherein the distance between opposing sides of the tissue wall of the LAA are larger than the LAA ostium. This allows the tissue wall of the LAA to be attached to the wall of the left atrium surrounding the LAA ostium, thereby closing the LAA. In other words, the closure anchors secure the LAA tissue to the left atrial wall, thereby closing the LAA predominantly with its own tissue.

In some embodiments, the plurality of closure anchors can be attached to the first side of the expandable disk or to the second side of the expandable disk opposite the first side. In certain embodiments, the plurality of closure anchors may be attached to a securing ring that is separate from the expandable disk. The LAA closure devices disclosed herein can assume a compact state for delivery and an expanded state for deployment to close a LAA. In the compact state, the LAA closure device can be configured to fit within a sheath of a delivery system. In the expanded or deployed state, the expandable disk assumes a size and shape having an expanded diameter that is larger than a typical ostium of a LAA. The LAA closure device can be included in a kit along with a delivery system.

Described herein are also methods for closing the LAA with the disclosed LAA closure devices. The methods include everting the tissue wall either from outside of the LAA or within the left atrium using a delivery system (e.g., a rounded catheter tip). The methods also include anchoring the device to the everted tissue with a deployment anchor. The methods also include releasing an expandable disk and expanding the disk to modify the shape of the everted LAA such that it covers the LAA ostium. The methods also include deploying closure anchors and engaging the closure anchors to the left atrial wall and the everted LAA tissue to secure the everted LAA tissue to the left atrial wall thereby closing the LAA predominantly with its own tissue. The methods also include disengaging and withdrawing the delivery system.

### Examples of LAA Closure Devices

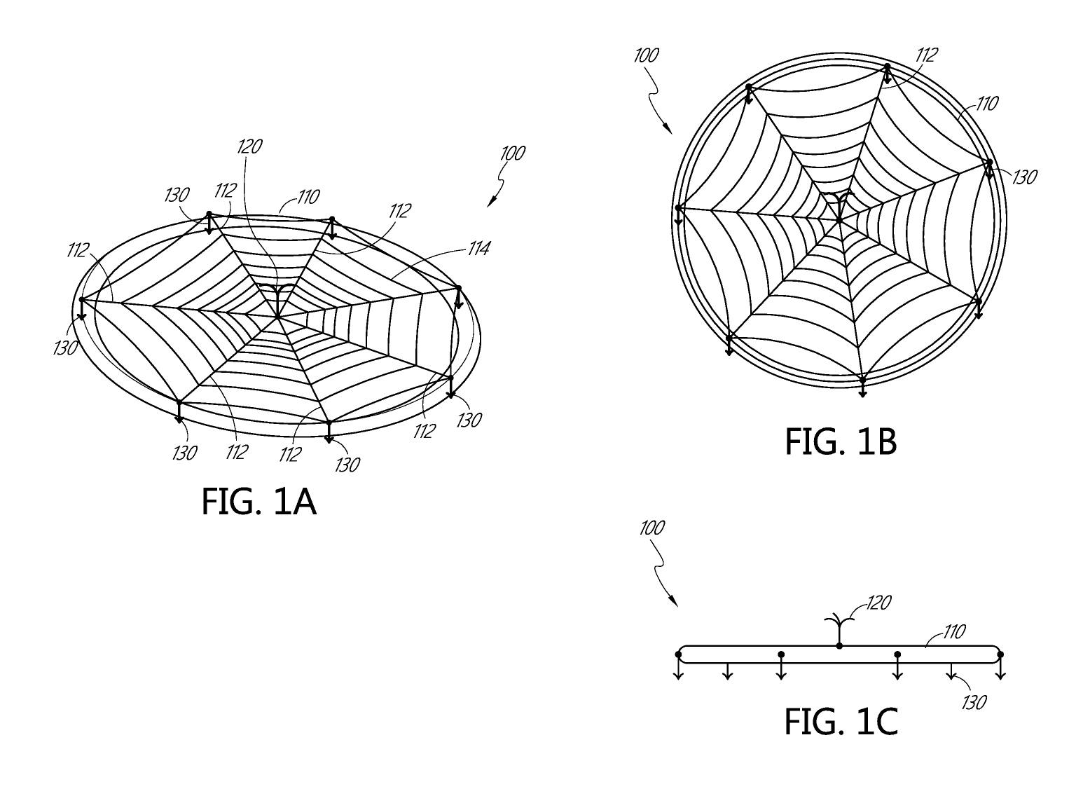

FIGS. 1A-1C illustrate various views of an example LAA closure device 100 that includes an expandable disk 110, a central or deployment anchor 120, and a plurality of peripheral or closure anchors 130. The LAA closure device 100 can be used to close a LAA of a patient using predominantly the tissue within the heart of the patient. The LAA closure device 100 is configured for use employing an external approach in closing the LAA of the patient.

The expandable disk 110 of the LAA closure device 100 can be a mesh or braided material and may be made of a self-expanding material. The expandable disk 110 is configured to assume a compact state when being delivered to the LAA site of the patient and to expand to a deployed state to close the LAA of the patient. The expandable disk 110 includes a plurality of radial supports 112 that are configured to provide structural support to the expandable disk 110. The expandable disk 110 can include 2 or more radial supports 112, 3 or more radial supports 112, 4 or more radial supports 112, 5 or more radial supports 112, 6 or more radial supports 112, or 7 or more radial supports 112. In some embodiments, the radial supports 112 emanate from a central location of the expandable disk 110. In some embodiments, the radial supports 112 can have different configurations such that they do not necessarily emanate from a central location of the expandable disk 110 but can have different geometries. The expandable disk 110 can also include auxiliary supports 114 connecting the radial supports 112 to provide additional structural support for the expandable disk 110. The auxiliary supports 114 can be concentric, can spiral around the expandable disk 110, and/or can provide a mesh or braided structure of the expandable disk 110.

The self-expanding disk 110 is configured to be posed in two positions, a compact position where the cross-section of the expandable disk 110 is small to permit delivery within a delivery system, and a deployed position where the expandable disk 110 is extended radially by forces exerted from within (e.g., by a deployment mechanism) or self-expanded (e.g., due to the use of shape memory alloys) to expand the everted LAA within the left atrium of the patient to close the LAA of the patient. The radial supports 112 and the auxiliary supports 114 can provide some or all of the forces that expand the expandable disk 110. In some embodiments, the delivery system includes one or more components that provides some or all the forces that expand expandable disk 110.

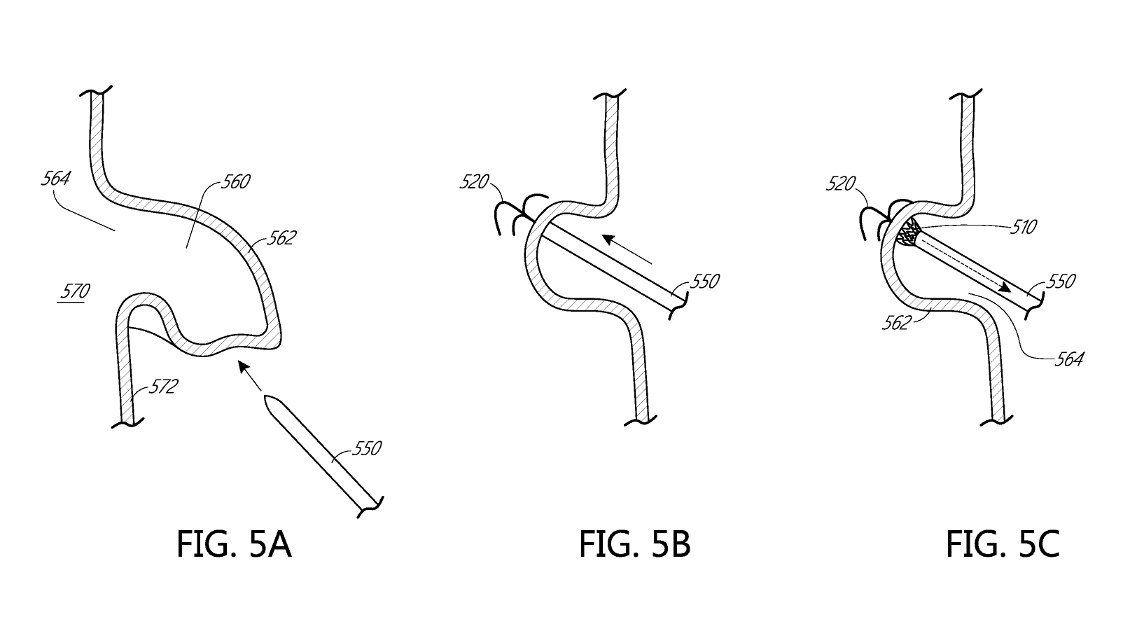

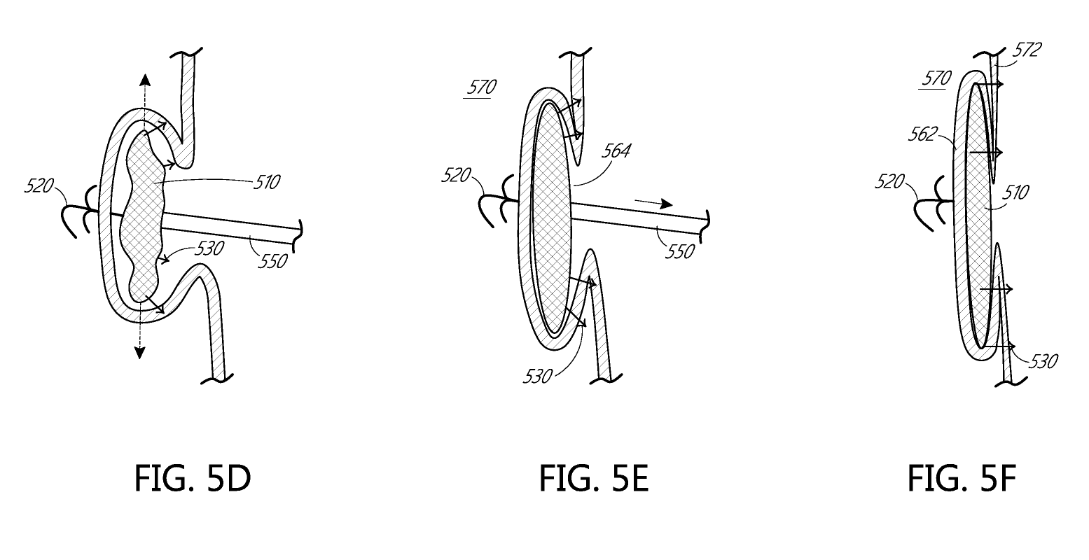

The expandable disk 110 can be configured to change size (e.g., collapse and expand) to allow the LAA closure device 100 to be implanted in an everted LAA of a patient to close the LAA. The expandable disk 110 can be made from plastically-expandable materials, shape memory alloys such as nickel titanium (nickel titanium shape memory alloys, or NiTi, as marketed, for example, under the brand name Nitinol), or other biocompatible metals. The radial supports 112 and/or the auxiliary supports 114 can be made of nickel titanium wires and/or braids. Accordingly, the expandable disk 110 can be made of nickel titanium wires and/or braids. The LAA closure device 100 with the expandable disk 110 can be suitable for crimping into a narrow configuration for installation and expandable to a wider, deployed configuration to flatten and close the LAA as described in greater detail herein with reference to FIGS. 5A-7F.

In certain implementations, the expandable disk 110 can include plastically-expandable materials that permit crimping of the LAA closure device 100 to a smaller profile for delivery and expansion of the LAA closure device 100 using a delivery system. In various implementations, the expandable disk 110 can include self-expanding material such as a shape memory alloy. This self-expanding LAA closure device 100 can be crimped to a smaller profile and held in this compact state with a restraining device such as a sheath of a delivery system. When the expandable disk 110 is positioned within an everted LAA, the restraining device is removed to allow the expandable disk 110 to self-expand to its expanded, deployed size. For example, LAA closure devices 100 can be crimped to a compressed state and introduced in the compressed state to the LAA using a delivery system (e.g., a catheter having a rounded tip) from an external approach where the delivery system everts the LAA and positions the LAA closure device 100 in a compact state within the everted LAA and then deploys the LAA closure device 100 so that it expands to a functional size to flatten and close the LAA.

In some embodiments, the expandable disk 110 is constructed with materials so that it can be radially compressed into a compressed or compact state for delivery, and can self-expand to a natural, uncompressed or functional state having a preset or targeted size or diameter. In certain implementations, the expandable disk 110 can assume a generally circular shape in its expanded form, however other shapes are possible and are considered within the scope of the present disclosure, such as, for example, elliptical shapes, oval shapes, irregular shapes, or the like. Accordingly, the targeted size or diameter the expandable disk 110 can refer to a diameter of a circle or an average or characteristic distance across the expanded disk 110 regardless of the exact shape of the disk 110. The targeted diameter of the expandable disk 110 can be such that the expandable disk has a larger diameter than a typical LAA ostium. For example, the targeted diameter of the expandable disk 110 can be at least about 10 mm and/or less than or equal to about 70 mm, at least about 15 mm and/or less than or equal to about 65 mm, at least about 20 mm and/or less than or equal to about 60 mm, at least about 25 mm and/or less than or equal to about 55 mm, at least about 30 mm and/or less than or equal to about 50 mm, or at least about 35 mm and/or less than or equal to about 45 mm.

The expandable disk 110 expands or tends toward a targeted diameter when free of external forces. In some embodiments, the expandable disk 110 expands or tends toward the targeted diameter in the presence of external forces such as when deployed within an everted LAA. The targeted diameter of the expandable disk 110 is configured to be larger than a typical LAA ostium so that when expanded within an everted LAA the LAA flattens against the left atrial wall covering the LAA ostium.

The expandable disk 110 includes a deployment anchor 120 and a plurality of securing anchors 130 to penetrate the native tissue at the targeted location to secure the LAA closure device 100 in place. The deployment anchor 120 and/or the plurality of closure anchors 130 can be any suitable projection from the expandable disk 110 such as, for example, hooks, barbs, anchors, or the like. In some embodiments, the deployment anchor 120 is made of a similar self-expanding material as the expandable disk 110. Similarly, the plurality of closure anchors 130 can be made of a similar self-expanding material as the expandable disk 110.

The deployment anchor 120 can be configured to penetrate or puncture the tissue of the LAA. The deployment anchor 120 can be attached to the expandable disk 110 at or near a central location of the expandable disk. The deployment anchor 120 can extend at least about 5 mm and/or less than or equal to about 15 mm from the expandable disk 110. In some embodiments, the deployment anchor 120 includes at least 3 arms and/or less than or equal to 6 arms. In certain implementations, the deployment anchor 120 includes 3 arms, 4 arms, 5 arms, or 6 arms. The arms of the deployment anchor 120 can be made of a self-expanding material such as nickel titanium.

The plurality of closure anchors 130 can be configured to penetrate or puncture the tissue of the LAA and the left atrial wall to secure the expandable disk 110 to the LAA and the left atrial wall. Individual closure anchors 130 can be attached to the expandable disk 110 at or near the ends of corresponding radial supports 112.

The LAA closure device 100 is configured with the deployment anchor 120 on a first side of the expandable disk and the plurality of closure anchors 130 on a second side of the expandable disk 110, the second side being opposite the first side. In this configuration, the LAA closure device 100 is configured for installation using an external approach to the heart. As described in greater detail herein with reference to FIGS. 5A-5F, the LAA closure device 100 is configured to be installed by everting the LAA using an external approach such that the deployment anchor 120 pierces the tissue of the LAA to secure the expandable disk 110 to the LAA and the plurality of closure anchors 130 are configured to pierce the LAA tissue and secure themselves to the tissue of the left atrial wall from within the everted LAA, thereby closing the LAA.

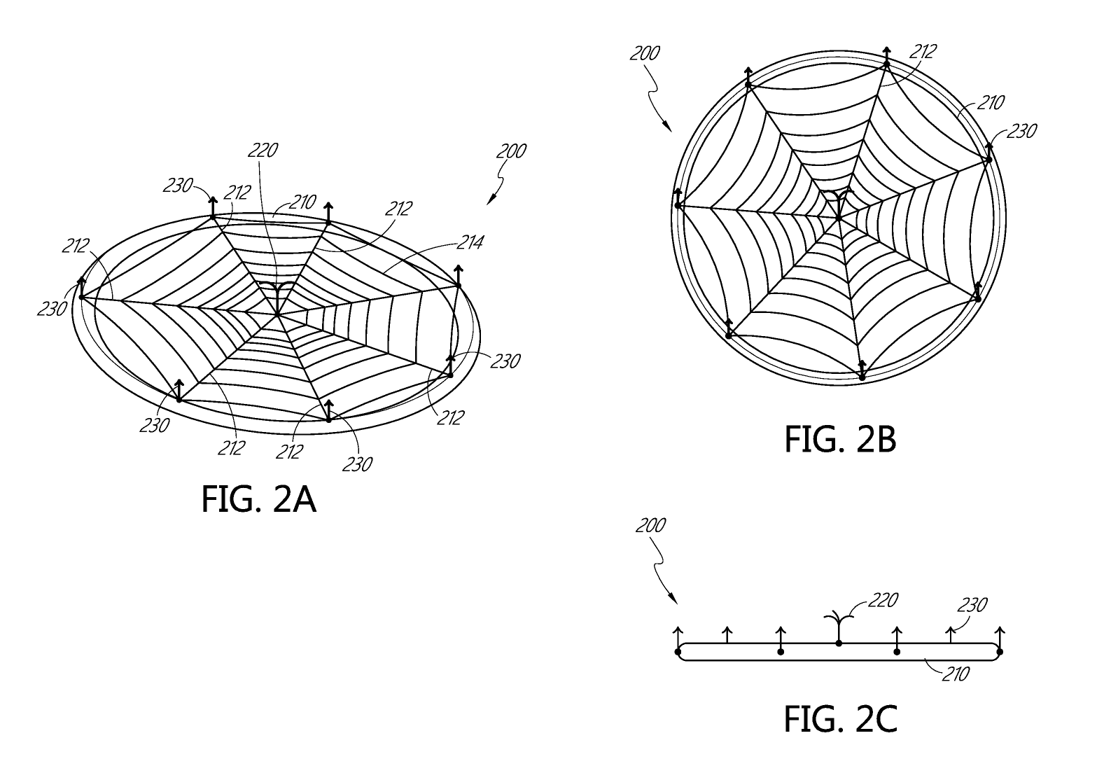

FIGS. 2A-2C illustrate various views of another example LAA closure device 200 that includes an expandable disk 210 having radial supports 212 and auxiliary supports 214, a central or deployment anchor 220, and a plurality of peripheral or closure anchors 230, similar to the LAA closure device 100. However, in contrast to the LAA closure device 100, the LAA closure device 200 is configured for an internal approach in closing the LAA of the patient.

The deployment anchor 220 of the LAA closure device 200 is attached to the expandable disk 210 on a first side of the expandable disk 210. The plurality of closure anchors 230 is attached to the expandable disk 210 on the first side of the expandable disk 210 such that the deployment anchor 220 and the plurality of closure anchors 230 are attached to the same side of the expandable disk 210.

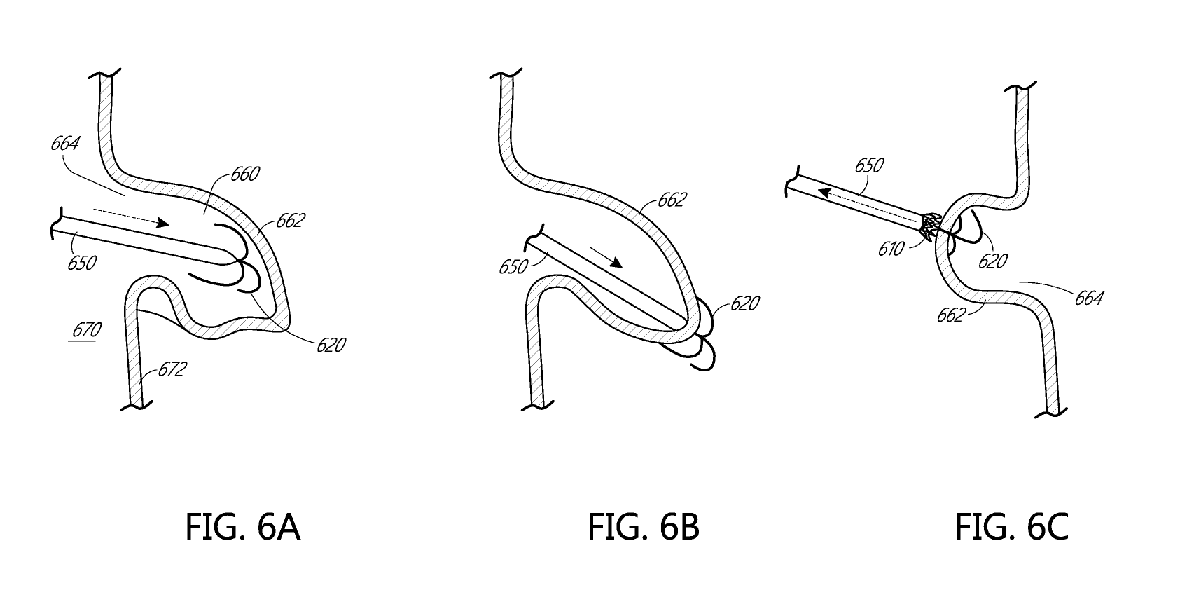

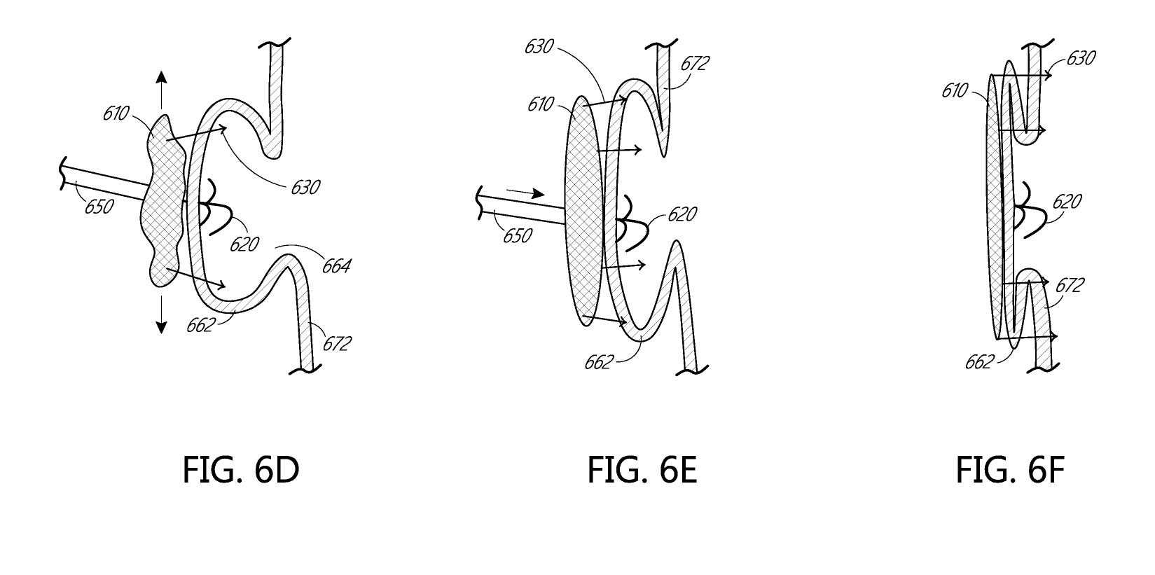

As described in greater detail herein with reference to FIGS. 6A-6F, the LAA closure device 200 is configured to be installed by everting the LAA using an internal approach such that the deployment anchor 220 pierces the tissue of the LAA to secure the expandable disk 210 to the LAA to facilitate eversion of the LAA and the plurality of closure anchors 230 are configured to pierce the LAA tissue and secure themselves to the tissue of the left atrial wall outside of the everted LAA to close the LAA.

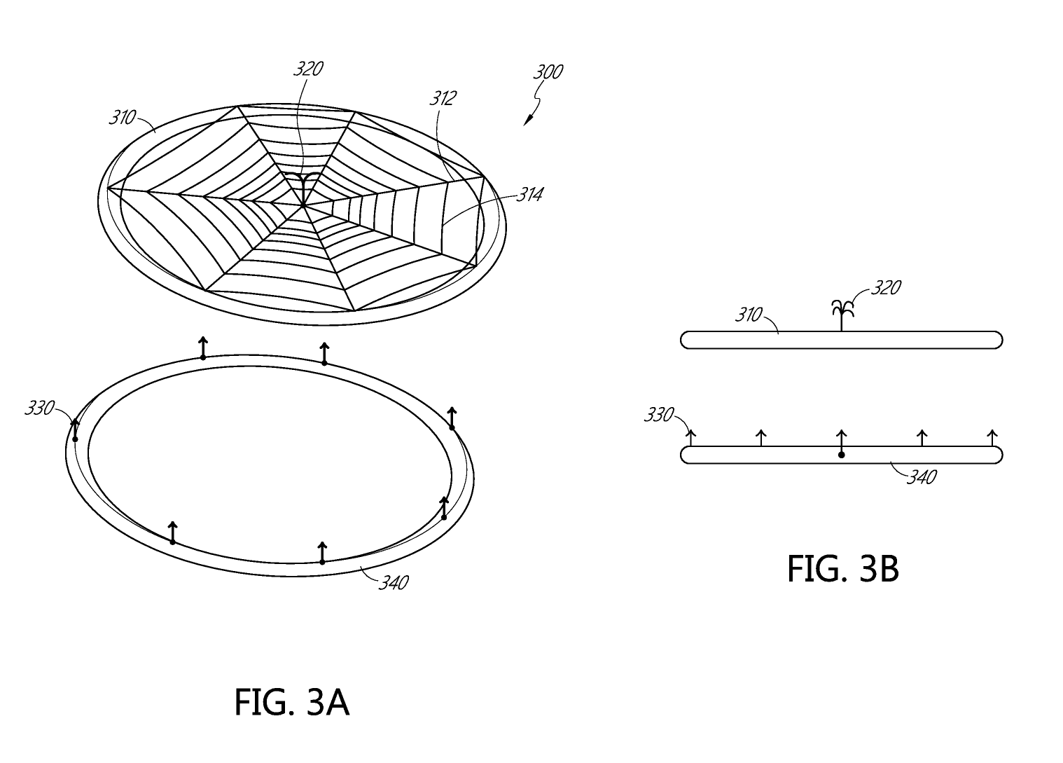

FIGS. 3A and 3B illustrate various views of another example LAA closure device 300 that includes an expandable disk 310 having radial supports 312 and auxiliary supports 314, and a central or deployment anchor 320, similar to the LAA closure devices 100 and 200. However, the LAA closure device 300 includes a securing ring 340 with the plurality of peripheral or closure anchors 130 attached thereto. The LAA closure device 300 is configured to close the LAA of the patient using a combination of internal and external approaches.

The securing ring 340 can be made of an expandable material similar to the expandable disk 310. The securing ring 340 can have a generally annular shape. The diameter of the securing ring 340 can be approximately the same as the diameter of the expandable disk 310. The diameter of the securing ring 340 can be larger than a diameter of the LAA ostium which is typically about 15 mm to about 30 mm in diameter. In some embodiments, the diameter of the securing ring 340 is larger than the diameter of the expandable disk 310. In certain embodiments, the diameter of the securing ring 340 is smaller than the diameter of the expandable disk 310 but still larger than the diameter of the LAA ostium. In certain implementations, the securing ring 340 can be a solid object (e.g., a disk or plate) rather than an annular one.

The securing ring 340 can be configured to be delivered in a compact state within a delivery system and to expand to a deployed state, similar to the expandable disk 310. In some embodiments, the LAA closure device 300 utilizes a delivery system having an external component with the expandable disk 310 and an internal component with the securing ring 340. In such embodiments, the delivery system can use the external component with the expandable disk 310 to evert the LAA and to expand the LAA to cover the LAA ostium and can use the internal component with the securing ring 340 to secure the LAA to the left atrial wall, thereby closing the LAA.

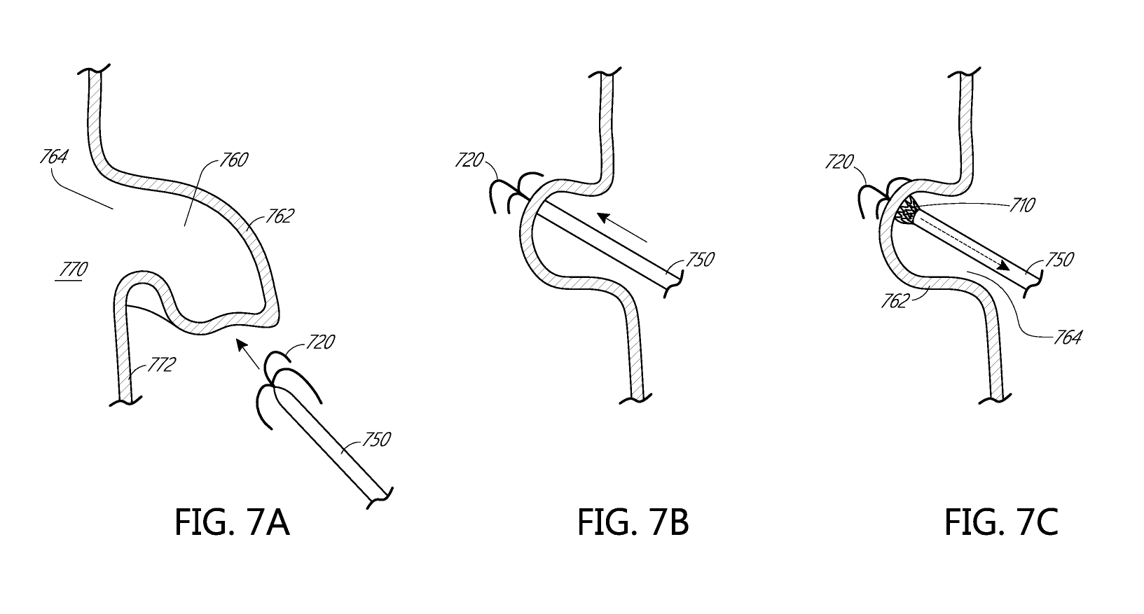

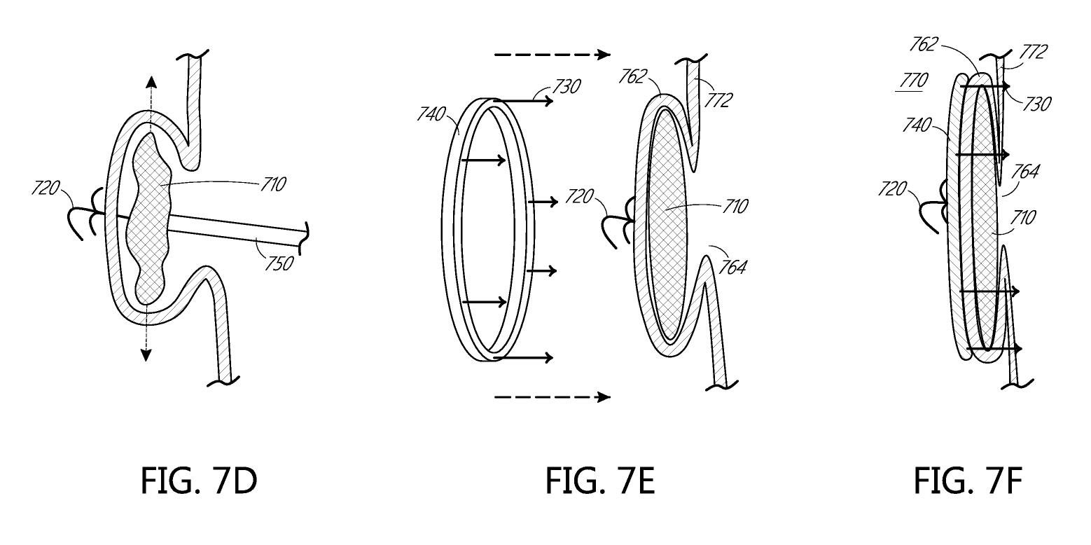

As described in greater detail herein with reference to FIGS. 7A-7F, the LAA closure device 300 is configured to be installed by everting the LAA using an external approach, as with the LAA closure device 100. The deployment anchor 320 pierces the tissue of the LAA to secure the expandable disk 310 to the LAA so that the expandable disk 310 opens within the everted LAA. The LAA closure device 300 is also configured to secure the LAA closed using an internal approach, as with the LAA closure device 200. The securing ring 340 with the securing anchors 330 is introduced from within the left atrium of the patient to secure the tissue of the LAA to the left atrial wall.

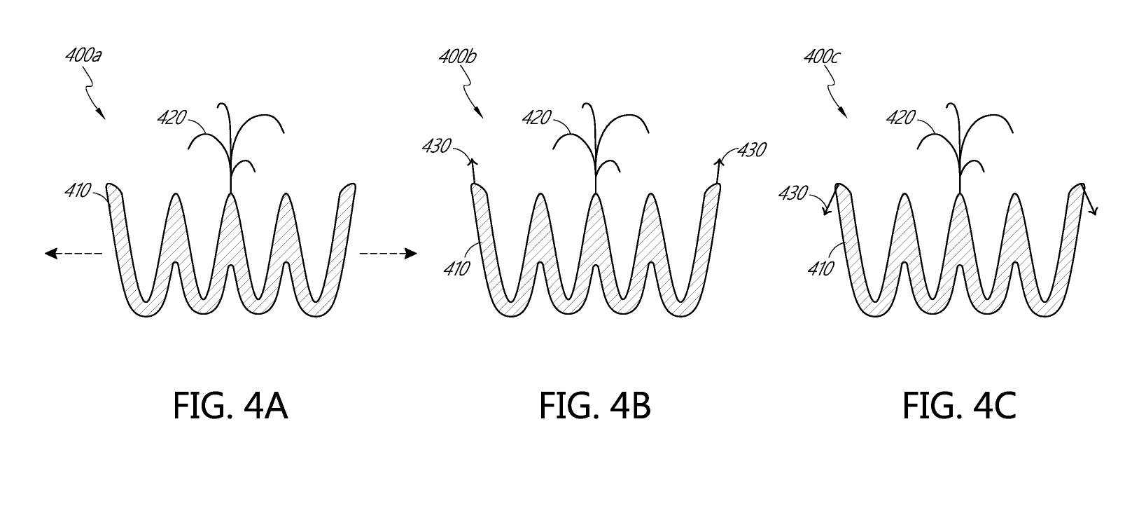

FIGS. 4A-4C illustrate various examples of LAA closure devices in a compact, collapsed, or crimped state. The LAA closure device 400a includes an expandable body 410 and a deployment anchor 420 attached to the expandable body 410. The LAA closure device 400b includes an expandable body 410, a deployment anchor 420 attached to the expandable body 410, and a plurality of securing anchors 430 configured to face in the same direction as the deployment anchor 420 in its expanded or deployed state. The LAA closure device 400c includes an expandable body 410, a deployment anchor 420 attached to the expandable body 410, and a plurality of securing anchors 430 configured to face in the opposite direction as the deployment anchor 420 in its expanded or deployed state.

The expandable body 410 can be configured to be crimped or collapsed to fit within a delivery system. The expandable body 410 remains in the crimped or collapsed state while the delivery system restricts the expandable body 410. Once the restriction is removed, the expandable body 410 can be configured to expand. In some embodiments, the delivery system includes one or more components that assist the expandable body 410 in assuming its deployed state. In certain embodiments, the expandable body 410 includes self-expanding material and the delivery system does not include any components that assists the expandable body 410 in expanding to a deployed state. In various implementations, the expandable body can be crimped or collapsed similar to an umbrella to facilitate expansion during deployment.

Expandable body 410 can be configured so that the deployment anchor 420 and the periphery of the expandable body 410 contact the tissue of the LAA while in a crimped or collapsed state. For implementations where the LAA closure device is to be deployed using an external approach (e.g., closure device 400a or 400c), such configurations ensure that the expandable body 410 is within the everted LAA upon deployment of the expandable body 410 so that expansion of the expandable body 410 causes the LAA to flatten to cover the LAA ostium from within the left atrium. For implementations where the LAA closure devices are to be deployed using an internal approach (e.g., closure device 400b), such configurations ensure that the securing anchors 430 contact the tissue of the LAA so that expansion of the expandable body 410 causes the LAA to flatten to cover the LAA ostium from within the left atrium.

### Implantation of LAA Closure Devices

FIGS. 5A-5F illustrate an example process for closing a LAA of a patient using an external approach. The illustrated process can use, for example, the LAA closure device 100 described herein with reference to FIGS. 1A-1C. By way of overview, the illustrated process uses a delivery system 550 to evert a LAA 560 by pushing against a LAA wall 562 until the LAA wall 562 passes through a LAA ostium 564. The delivery system 550 then retracts a sheath covering an expandable body 510 of a LAA closure device, allowing the expandable body 510 to expand. The expandable body 510 is then pulled back using the delivery system 550 to cause closure anchors 530 to secure the flattened and everted LAA 560 to the left atrial wall 572. In addition, FIGS. 5A-5F illustrate components of a LAA closure kit that includes the delivery system 550 and a LAA closure device, such as the LAA closure device 100 described herein with reference to FIGS. 1A-1C.

FIG. 5A illustrates a delivery system 550 approaching a LAA 560 having a LAA wall 562 and a LAA ostium 564. The delivery system 550 is being introduced using an external approach such that the delivery system is external to the left atrium 570. In some embodiments, the delivery system 550 includes a rounded catheter tip.

FIG. 5B illustrates the delivery system 550 everting the LAA. Upon everting the LAA, the delivery system 550 deploys a deployment anchor 520 of a LAA closure device. The deployment anchor 520 pierces the LAA wall 562 to secure a LAA closure device to the LAA wall 562.

FIG. 5C illustrates the delivery system 550 being retracted to release expandable body 510 of a LAA closure device. With the deployment anchor 520 secured to the LAA wall 562, retraction of the delivery system 550 can remove a sheath or other component that restricts the expandable body 510 to maintain it in a collapsed state, allowing the expandable body 510 to begin to expand.

FIG. 5D illustrates the expandable body 510 expanding within the everted LAA. Upon expanding, the expandable body 510 pushes against the walls of the everted LAA to flatten the LAA 560, as indicated by the dashed arrows. Furthermore, upon expanding the plurality of closure anchors 530 deploy.

FIG. 5E illustrates the expandable body 510 in a fully expanded state. The expandable body 510 expands to a diameter greater than a width of the opening of the LAA ostium 564. In the fully expanded state and with the closure anchors 530 deployed, the delivery system 550 applies a force on the expandable body 530 to cause the closure anchors 530 to pierce the LAA wall 562 and to pierce the left atrial wall 572. Applying this force causes the LAA closure device to close the LAA 560 within the left atrium 570 by securing it in a flattened state to the left atrial wall 572.

FIG. 5F illustrates the LAA closure device installed in the everted LAA 560 with the deployment anchor secured to the LAA wall 562 and the plurality of closure anchors 530 securing the expandable body 510 to the left atrial wall 572. The delivery system 550 is configured to disengage from the LAA closure device after the device secures an everted LAA 560 to a left atrial wall 572.

FIGS. 6A-6F illustrate an example process for closing a LAA of a patient using an internal approach. The illustrated process can use, for example, the LAA closure device 200 described herein with reference to FIGS. 2A-2C. By way of overview, the illustrated process uses a delivery system 650 to evert a LAA 660 by pulling a LAA wall 662 until the LAA wall 662 passes through a LAA ostium 664. The delivery system 650 then retracts a sheath covering an expandable body 610 of a LAA closure device, allowing the expandable body 610 to expand. The expandable body 610 is then pushed forward using the delivery system 650 to cause closure anchors 630 to engage the LAA tissue wall 662. Once expanded, additional force is applied to the expandable body 610 to secure the flattened and everted LAA 660 to the left atrial wall 672. In addition, FIGS. 6A-6F illustrate components of a LAA closure kit that includes the delivery system 650 and a LAA closure device, such as the LAA closure device 200 described herein with reference to FIGS. 2A-2C.

FIG. 6A illustrates a delivery system 650 approaching a LAA 660 having a LAA wall 662 and a LAA ostium 664. The delivery system 650 is being introduced using an internal approach such that the delivery system 650 is within the left atrium 670. In some embodiments, the delivery system 650 includes a rounded catheter tip.

FIG. 6B illustrates the delivery system 650 everting the LAA 660 by piercing the LAA tissue wall 662 with a deployment anchor 620 that secures the expandable body 610 and the delivery system to the LAA tissue wall 662. Once secured, a force is applied (e.g., by pulling) on the delivery system 650 toward the left atrium 670 to evert the LAA 660.

FIG. 6C illustrates the delivery system 650 being retracted to release the expandable body 610 of a LAA closure device. With the deployment anchor 620 secured to the LAA wall 662, retraction of the delivery system 650 can remove a sheath or other component that restricts the expandable body 610 to maintain it in a collapsed state, allowing the expandable body 610 to begin to expand.

FIG. 6D illustrates the expandable body 610 expanding while in contact with or in close proximity to the everted LAA 660. Upon expanding, the plurality of closure anchors 630 deploy and begin to secure the expandable body 610 to the LAA wall 662. In addition, the expandable body 610 flattens the LAA 660 with the help of the closure anchors 630, as indicated by the arrows.

FIG. 6E illustrates the expandable body 610 in a fully expanded state. The expandable body 610 expands to a diameter greater than a diameter of the opening of the LAA ostium 664. In the fully expanded state and with the closure anchors 630 deployed, the delivery system 650 applies a force on the expandable body 610 toward the left atrial wall 672 to cause the closure anchors 630 to pierce the LAA wall 662 and to pierce the left atrial wall 672. Applying this force causes the LAA closure device to close the LAA 660 within the left atrium 670.

FIG. 6F illustrates the LAA closure device installed in the everted LAA 660 with the deployment anchor secured to the LAA wall 662 and the plurality of closure anchors 630 securing the expandable body 610 to the LAA wall 662 and the left atrial wall 672. The delivery system 650 is configured to disengage from the LAA closure device after the device secures an everted LAA 660 to a left atrial wall 672.

FIGS. 7A-7F illustrate an example process for closing a LAA of a patient using a combination of an internal and an external approach. The illustrated process can use, for example, the LAA closure device 300 described herein with reference to FIGS. 3A-3C. By way of overview, the illustrated process uses an external component of a delivery system 750 to evert a LAA 760 by pushing against a LAA wall 762 until the LAA wall 762 passes through a LAA ostium 764. The external component of the delivery system 750 then retracts a sheath covering an expandable body 710 of a LAA closure device, allowing the expandable body 710 to expand. A securing ring 740 is deployed within the left atrium 770 using an internal component of the delivery system 750. The securing ring 740 is pushed toward the everted LAA 760 to cause closure anchors 730 to secure the flattened and everted LAA 760 to the left atrial wall 772. In addition, FIGS. 7A-7F illustrate components of a LAA closure kit that includes a LAA closure device, such as the LAA closure device 300 described herein with reference to FIGS. 3A-3C, and the delivery system 750 having an internal component for the securing ring 740 and an external component for the expandable body 710.

FIG. 7A-7C illustrate a portion of the installation process that is similar to the portion of the process described with reference to FIGS. 5A-5C. In particular, a delivery system 750 is introduced to evert a LAA 760. A deployment anchor 720 pierces and attaches to the LAA wall 762. A sheath of the deployment system 750 is retracted to allow the expandable body 710 to be deployed.

The step of the process illustrated in FIG. 7D is similar to the one illustrated in FIG. 5D with the notable exception that upon expanding, the expandable body 710 does not deploy a plurality of closure anchors 730. However, the expandable body 710 does cause the everted LAA 760 to begin to flatten and to cover the LAA ostium 764.

FIG. 7E illustrates the expandable body 710 in a fully expanded state. The expandable body 710 expands to a diameter greater than a diameter of the opening of the LAA ostium 764. In the fully expanded state, an internal component of the delivery system 750 introduces the securing ring 740 having the closure anchors 730. The internal component of the delivery system applies a force on the securing ring 740 to cause the closure anchors 730 to pierce the LAA wall 762 and to pierce the left atrial wall 772. Applying this force causes the LAA closure device to close the LAA 760 within the left atrium 770.

FIG. 7F illustrates the LAA closure device installed in the everted LAA 760 with the deployment anchor 720 secured to the LAA wall 762 and the plurality of closure anchors 730 securing the securing ring 740 and the expandable body 710 to the left atrial wall 772, the securing ring 740 being within the left atrium 770 and the expandable body 710 being within the everted LAA 760. The delivery system 750 is configured to disengage from the LAA closure device after the device secures an everted LAA 760 to a left atrial wall 772.

The mechanism of closing the LAA using the disclosed closure devices and processes results in the LAA being closed predominantly using the tissue of the LAA and the left atrium. Accordingly, the devices and processes described with reference to FIGS. 5A-5F, 6A-6F and 7A-7F advantageously reduce the chances of clotting due at least in part to reducing or minimizing foreign bodies in the flow path of the left atrium. Furthermore, this advantageously reduces the chances of clotting due at least in part to the elimination of the LAA through eversion, flattening, and attachment to the left atrium. In addition, the disclosed LAA closure devices and processes for installation of said devices allows for LAA closure irrespective of the shape of the LAA. In other words, the disclosed devices, systems, processes, and methods can be configured to close a LAA having a wide variety of configurations, sizes, and shapes.

### Methods of Implanting LAA Closure Devices

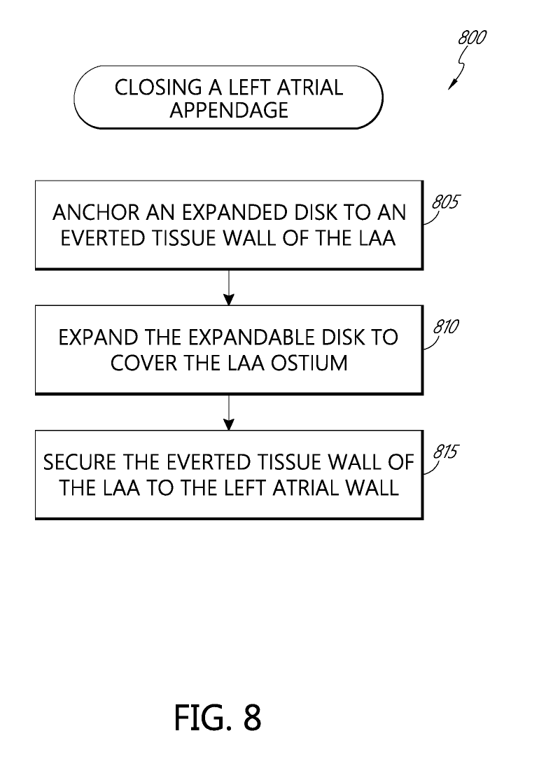

FIG. 8 illustrates a flow chart of an example method 800 of closing a left atrial appendage. The method 800 can be performed using any suitable LAA closure device, such as the devices described herein with reference to FIGS. 1A-3C. The method 800 is advantageous because it can be performed using an internal approach, an external approach, or a combination of internal and external approaches. Thus, the method 800 can be utilized in conjunction with minimally invasive surgery or open surgery. In addition, the method 800 advantageously closes the left atrial appendage predominantly using the tissue of the left atrial appendage and left atrium, reducing the probability of clots. Furthermore, the method 800 advantageously functions to close the LAA substantially irrespective of the shape and/or size of the LAA.

In step 805, a deployment system and a LAA closure device anchor an expandable disk to an everted tissue wall of the LAA. The LAA closure device includes the expandable disk with a connected deployment anchor that pierces or otherwise attaches to the tissue of the LAA. In some embodiments, the deployment anchor first pierces the tissue wall of the LAA and can assist or facilitate with everting the LAA. In certain embodiments, the deployment anchor pierces the tissue wall of the LAA after the LAA has been everted with a deployment system (e.g., a rounded tip catheter). In various implementations, the expandable disk is anchored to the LAA by applying a force on the deployment system directed from outside of the heart toward the left atrium. In certain implementations, the expandable disk is anchored to the LAA by applying a force on the deployment system directed from within the left atrium toward the LAA.

In some embodiments, everting the tissue wall includes using a rounded catheter tip from a location external to the heart to evert the left atrial appendage. In certain embodiments, everting the tissue wall includes using a rounded catheter tip from a location within the left atrium to evert the LAA.

In block 810, an expandable disk expands the everted tissue wall to cover an ostium of the LAA. The deployment system can include a sheath that restricts the expandable disk until the sheath is retracted. Upon retraction of the sheath, the expandable disk can expand to flatten the LAA to cover the ostium. In some embodiments, the expandable disk flattens the LAA from within the everted LAA. In certain embodiments, the expandable disk flattens the LAA from within the left atrium but outside of the everted LAA. In various implementations, closure anchors attached to the expandable disk aid in flattening the LAA.

In block 815 the LAA closure device secures the everted tissue wall of the left atrial appendage to a wall of the left atrium. The expandable disk can include a plurality of closure anchors that pierce or otherwise attach to the LAA tissue wall and the left atrial wall. In this way, the LAA is closed predominantly using tissue of the heart. In some embodiments, the deployment system is used to apply a force on the expandable disk after it has expanded to cause the closure anchors to pierce the LAA wall and anchor themselves into the left atrial wall. The force applied on the expandable disk can be applied by the deployment system from outside of the heart or from within the left atrium. In certain embodiments, the deployment system is used to apply a force on a securing ring that is within the left atrium to cause the closure anchors to pierce the LAA wall and anchor themselves into the left atrial wall. The force applied on the securing ring is applied by the deployment system from within the left atrium.

### Additional Embodiments

As used herein, the terms “collapsible,” “expandable,” and other related words are used interchangeably to indicate that the disclosed structures can change their radial size to become smaller for delivery (e.g., a collapsed, compact, or crimped state) and to become larger for implantation and operation in the heart (e.g., an expanded, functional, or deployed state). It should be understood that decreasing the radial size of the structure may increase, for example, its longitudinal dimension. However, for the purposes of this disclosure, this is still considered to be collapsible.

As used herein, the terms “evert,” “invaginate,” “invert” and other related words are used interchangeably to indicate that the left atrial appendage is turned inside out by either pushing or pulling the left atrial appendage through its ostium so that the left atrial appendage is within the left atrium. The result of this eversion or invagination is that the portion of the left atrial appendage wall that previously was external to the heart prior to eversion is within the left atrium after eversion.

Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described herein. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

Reference throughout this specification to “certain embodiments” or “an embodiment” means that a particular feature, structure or characteristic described in connection with the embodiment is included in at least some embodiments. Thus, appearances of the phrases “in some embodiments” or “in an embodiment” in various places throughout this specification are not necessarily all referring to the same embodiment and may refer to one or more of the same or different embodiments. Furthermore, the particular features, structures or characteristics can be combined in any suitable manner, as would be apparent to one of ordinary skill in the art from this disclosure, in one or more embodiments.

It should be appreciated that in the above description of embodiments, various features are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

What is claimed is:

1. A device for closing a left atrial appendage, the device comprising:
an expandable disk having an expanded diameter that is larger than 10 mm;
a deployment anchor attached to the expandable disk near a center of a first side of the expandable disk, the deployment anchor configured to puncture a tissue of a left atrial appendage and to anchor the expandable disk to the tissue of the left atrial appendage; and
a plurality of closure anchors attached to the expandable disk near a periphery of the expandable disk, the plurality of closure anchors configured to secure the expandable disk to tissue of a left atrium,
wherein the device is configured for delivery in a compact state and expands to an expanded state to cause an everted left atrial appendage to assume a size that is larger than an ostium of the left atrial appendage.

2. The device of claim 1 wherein the closure anchors are attached to the expandable disk on a second side of the expandable disk, the second side opposite the first side.

3. The device of claim 1 wherein the closure anchors are attached to the expandable disk on the first side of the expandable disk.

4. The device of claim 1 wherein the device is configured to assume a compact state for delivery and a deployed state for deployment for closing a left atrial appendage.

5. The device of claim 5 wherein the compact state comprises reducing a diameter of the expandable disk to fit within a sheath of a delivery system.

6. The device of claim 5 wherein the deployed state comprises the expandable disk assuming a size and shape such that the expanded diameter is larger than a typical ostium of a left atrial appendage.

7. The device of claim 1 wherein the expandable disk includes radial supports.

8. The device of claim 7 wherein individual closure anchors are coupled to ends of corresponding radial supports.

9. A left atrial appendage closure kit including the device of claim 1 and a delivery system having a retractable sheath configured to house the expandable disk in a compact state.

10. The kit of claim 9 wherein the delivery system the sheath is configured to be pulled back during operation to release the expandable disk in the compact state such that the expandable disk expands to assume a deployed state.

11. A method for closing a left atrial appendage, the method comprising:
anchoring an expandable disk to an everted tissue wall of the left atrial appendage with a deployment anchor attached to the expandable disk;
expanding the expandable disk to expand the everted tissue wall to cover an ostium of the left atrial appendage; and
securing the everted tissue wall of the left atrial appendage to a wall of the left atrium with a plurality of closure anchors.

12. The method of claim 11 further comprising everting the tissue wall of the left atrial appendage so that the tissue wall of the left atrial appendage is within the left atrium.

13. The method of claim 12 wherein everting the tissue wall comprises using a rounded catheter tip from a location external to a heart to evert the left atrial appendage.

14. The method of claim 12 wherein everting the tissue wall comprises using a rounded catheter tip from a location within the left atrium to evert the left atrial appendage.

15. The method of claim 11 wherein the method is performed during a minimally invasive procedure.

16. The method of claim 11 wherein the method is performed during open heart surgery.

17. A device for closing a left atrial appendage, the device comprising:
an expandable disk having an expanded diameter that is larger than 10 mm;
a deployment anchor attached to the expandable disk near a center of a first side of the expandable disk, the deployment anchor configured to puncture a tissue of a left atrial appendage and to anchor the expandable disk to the tissue of the left atrial appendage;
a securing ring forming an annulus; and
a plurality of closure anchors attached to the securing ring, the plurality of closure anchors configured to secure the expandable disk to tissue of a left atrium,
wherein the expandable disk is configured for delivery in a compact state and expands to an expanded state to cause an everted left atrial appendage to assume a size that is larger than an ostium of the left atrial appendage.

18. The device of claim 17 wherein the expandable disk includes radial supports.

19. The device of claim 17 wherein the plurality of closure anchors comprises less than or equal to 6 anchors.

20. The device of claim 17 wherein the plurality of closure anchors comprises at least 2 anchors.
